(19) United States
(12) Patent Application Publication
(10) Pub. No.: US 2021/0076983 A1
(43) Pub. Date: Mar. 18, 2021

(54) DETERMINATION DEVICE, DETERMINATION METHOD, AND NON-TRANSITORY RECORDING MEDIUM

(71) Applicant: CASIO COMPUTER CO., LTD., Tokyo (JP)

(72) Inventors: Takashi YAMAYA, Tokyo (JP); Kouichi NAKAGOME, Tokorozawa-shi (JP); Mitsuyasu NAKAJIMA, Tokyo (JP); Yasushi MAENO, Tokyo (JP)

(21) Appl. No.: 17/024,513

(22) Filed: Sep. 17, 2020

(30) Foreign Application Priority Data
Sep. 18, 2019 (JP) 2019-169556

(51) Int. Cl. A61B 5/11; A61B 5/00

(52) U.S. Cl. CPC A61B 5/1115; A61B 5/7465; A61B 2562/0219; A61B 5/002; A61B 5/742

(57) ABSTRACT

An acceleration acquirer of a controller of an in-bed and out-of-bed determination device acquires, in a time series, acceleration of a subject from a sensor that is an acceleration sensor attached by the subject, a body motion determiner determines, based on the acquired acceleration, whether there is a body motion of the subject at each time, an evaluator evaluates a distribution of the acceleration at a time at which it is determined that there is not a body motion, an estimator estimates a body axis of the subject based on the evaluated distribution of the acceleration, and an in-bed and out-of-bed determiner determines at least one of in bed and out of bed of the subject based on the estimated body axis of the subject that is estimated by the estimator.

### CROSS-REFERENCE TO RELATED APPLICATION

This application claims the benefit of Japanese Patent Application No. 2019-169556 filed on Sep. 18, 2019, the entire disclosure of which is incorporated by reference herein.

### FIELD

This application relates generally to a determination device, a determination method, and a non-transitory recording medium.

### BACKGROUND

Systems have been developed for ascertaining the sleep state of people in order to manage the health conditions and the like of people. In order to ascertain the health condition of a person, first, it must be determined if the person is in bed or out of bed. With regards to in bed/out of bed determination, if the direction of the axis of the body (body axis) is known, it is possible to determine, from a detection value of an acceleration sensor attached on the body, if the person is sitting/standing up or lying down based on the angle between the direction of gravity and the body axis. As a result, for example, it is possible to determine that the person is out of bed if the person is sitting/standing up, and determine that the person is in bed if no body motion is detected for a predetermined amount of time while the person is lying down. For example, Unexamined Japanese Patent Application Publication No. 2015-159850 discloses a sleep state evaluation device and the like in which a sensor is attached on the wrist of a person. With this sleep state evaluation device, the X axis, which is the longitudinal direction of the arm of the person, is assumed to be the body axis of the person, and the posture of the person is determined based on the angle of inclination of the X axis.

### SUMMARY

A determination device according to the present disclosure is a determination device including at least one processor, and the processor: acquires, in a time series, information about a body motion of a subject from a sensor attached to the subject; determines, based on the acquired information about the body motion, whether there is a body motion of the subject at each time, evaluates a distribution of the information about the body motion of the subject at a time at which it is determined that there is not a body motion, and estimates a body axis of the subject based on the evaluated distribution of the information about the body motion; and determines at least one of in bed and out of bed of the subject based on the estimated body axis of the subject.

Also, a determination method according to the present disclosure is a determination method including: acquiring, in a time series, information about a body motion of a subject; determining, based on the acquired information about the body motion, whether there is a body motion of the subject at each time, evaluating a distribution of the information about the body motion of the subject at a time at which it is determined that there is not a body motion, and estimating a body axis of the subject based on the evaluated distribution of the information about the body motion; and determining at least one of in bed and out of bed of the subject based on the estimated body axis of the subject.

Also, a non-transitory computer-readable recording medium according to the present disclosure is a non-transitory computer-readable recording medium on which a program is recorded, the program causing a computer of a determination device to: acquire, in a time series, information about a body motion of a subject; determine, based on the acquired information about the body motion, whether there is a body motion of the subject at each time, evaluate a distribution of the information about the body motion of the subject at a time at which it is determined that there is not a body motion, and estimate a body axis of the subject based on the evaluated distribution of the information about the body motion; and determine at least one of in bed and out of bed of the subject based on the estimated body axis of the subject.

### BRIEF DESCRIPTION OF THE DRAWINGS

A more complete understanding of this application can be obtained when the following detailed description is considered in conjunction with the following drawings, in which:

FIG. 1 is a drawing illustrating a configuration example of a determination device according to Embodiment 1;

FIG. 2 is a flowchart of body axis estimation processing according to Embodiment 1;

FIG. 3 is a flowchart of body motion determination processing according to Embodiment 1;

FIG. 4 is a drawing explaining a moving average of the norm of acceleration;

FIG. 5 is a flowchart of acceleration distribution evaluation processing according to Embodiment 1;

FIG. 6 is a drawing explaining the direct product of a matrix of accelerations;

FIG. 7 is a flowchart of determination processing according to Embodiment 1;

FIG. 8 is a flowchart of acceleration distribution evaluation processing according to Embodiment 2; and

FIG. 9 is a drawing explaining singular value decomposition of a matrix of accelerations.

### DETAILED DESCRIPTION

Hereinafter, embodiments are described while referencing the drawings. Note that, in the drawings, identical or corresponding components are marked with the same reference numerals.

### Embodiment 1

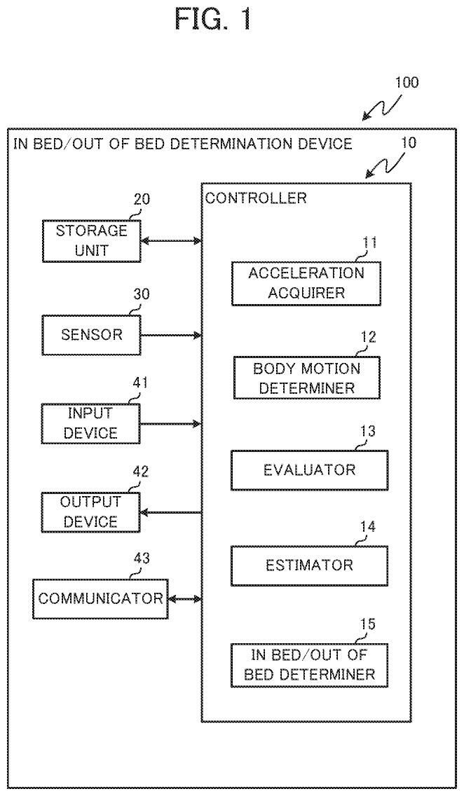

A determination device 100 according to Embodiment 1 detects a body motion (motion of the body) of a subject, estimates a body axis of the subject by evaluating a distribution of accelerations obtained from the detected body motion, and determines if the subject is in bed or out of bed. As illustrated in FIG. 1, the determination device 100 includes, as functional components, a controller 10, a storage unit 20, a sensor 30, an input device 41, an output device 42, and a communicator 43.

The controller 10 is configured from a central processing unit (CPU) includes a processor or the like. The controller 10 executes a program stored in the storage unit 20 to realize the functions of the hereinafter described components (an acceleration acquirer 11, a body motion determiner 12, an evaluator 13, an estimator 14, and a determiner 15). The controller 10 also includes a function for measuring time.

The storage unit 20 is configured from memory devices (read-only memory (ROM), random access memory (RAM), and the like). Programs to be executed by the CPU of the controller 10 and data needed in advance to execute these programs are stored in the ROM. Data that is created or modified during the execution of the program is stored in the RAM.

The sensor 30 includes an acceleration sensor for detecting the body motion of the subject. The acceleration sensor detects acceleration in the direction of each of three axes (mutually orthogonal X axis, Y axis, and Z axis) to detect the body motion of the subject. In the present embodiment, the acceleration sensor is attached on an ear of the subject.

The input device 41 is constituted by a keyboard, a mouse, a touch panel, or the like. In one example, the input device 41 is an interface for receiving user operations such as start/end instructions for body axis estimation processing and determination processing.

In one example, the output device 42 is constituted by a liquid crystal display (LCD), an electroluminescence (EL) display, or the like. In one example, the output device 42 displays determination results of the in bed/out of bed determination performed by the determination device 100.

The communicator 43 is a communication interface for exchanging data and the like with other external devices. This communication interface may be wireless or wired. In one example, the determination device 100 is capable of sending, via the communicator 43, the determination results of the in bed/out of bed determination and/or estimated body axis information to an external server or the like.

Next, the functional configuration of the controller 10 of the determination device 100 will be described. The controller 10 realizes the functions of an acceleration acquirer 11, a body motion determiner 12, an evaluator 13, an estimator 14, and a determiner 15, and estimates the direction of the body axis.

Using the acceleration sensor of the sensor 30, the acceleration acquirer 11 acquires the acceleration of the subject in a time series. Specifically, the acceleration acquirer 11 samples, at a predetermined sampling frequency (for example, 40 Hz), detection values (three-dimensional vectors including values of acceleration in the direction of each of three axes) obtained by the acceleration sensor to acquire an acceleration data string. Thus, the acceleration acquirer 11 functions as an acceleration acquisition device.

The body motion determiner 12 determines, based on the acceleration acquired by the acceleration acquirer 11, whether there is body motion of the subject at each time. Specifically, the body motion determiner 12 first calculates the norm of each acceleration from the acceleration at each time acquired by the acceleration acquirer 11. The body motion determiner 12 determines that there is no body motion if a value, obtained by removing the DC component from the norm of acceleration at each time, is less than or equal to a predetermined body motion determination threshold, and determines that there is body motion if the value is greater than the body motion determination threshold.

For the body motion determination threshold, an appropriate value is calculated and set based on experiments or the like such that it is determined that there is no body motion at times such as when the subject is breathing quietly, and it is determined that there is body motion when the subject is moving such as changing sleeping positions, or the like. For example, the appropriate value of the body motion determination threshold can be obtained by collating norms of acceleration, calculated from the values from the acceleration sensor obtained when the subject actually wore the acceleration sensor and slept, with the motions of the subject. Thus, the body motion determiner 12 functions as a body motion determination device.

The evaluator 13 evaluates the distribution of accelerations at times at which the body motion determiner 12 determines that there is no body motion. Specifically, the evaluator 13 extracts, from among the acceleration data string acquired by the acceleration acquirer 11, the accelerations at the times at which the body motion determiner 12 determines that there is no body motion, obtains the direct product of a matrix of the extracted accelerations, and performs an eigenvalue decomposition of this direct product, to obtain eigenvalues and the eigenvector corresponding to each of the eigenvalues. The eigenvalue and the eigenvector are values in which features (the distribution of acceleration) of the motion of the subject are reflected. Thus, the evaluator 13 functions as an evaluation device.

The estimator 14 estimates the direction of the body axis of the subject or the direction of gravity based on the distribution of acceleration evaluated by the evaluator 13. Specifically, the estimator 14 determines if there is a roll over, such as turning over, of the subject based on the magnitude of the eigenvalue calculated by the evaluator 13. If it is determined that there is a roll over, the estimator 14 estimates the direction vector in which the distribution of acceleration is small to be the direction of the body axis and, if it is determined that there is not a roll over, the estimator 14 estimates the direction in which the distribution of acceleration is great to be the direction of gravity. Thus, the estimator 14 functions as an estimation device.

The determiner 15 determines if the subject is in bed or out of bed based on the direction of the body axis (or gravity) estimated by the estimator 14, and the direction of acceleration acquired by the acceleration acquirer 11. Thus, the determiner 15 functions as a determination device.

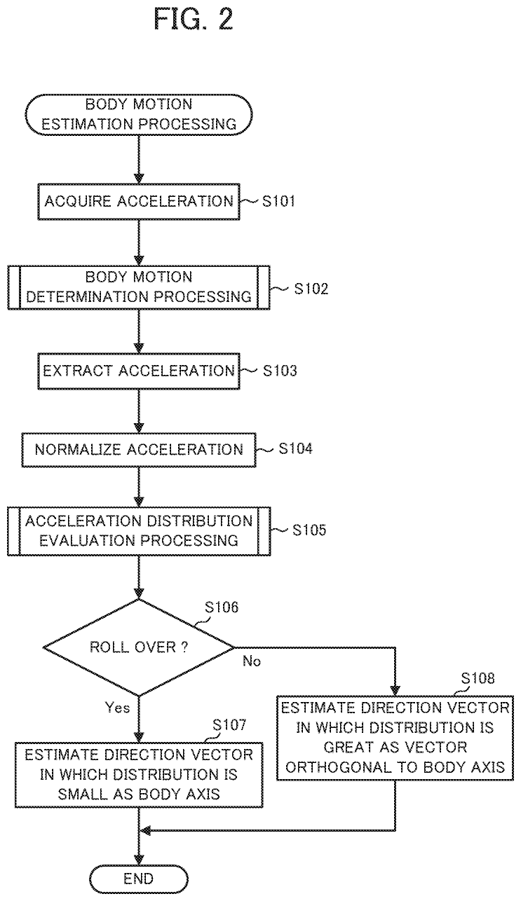

The functional configuration of the determination device 100 is described above. The determination device 100 must perform body axis estimation processing before performing determination processing. The body axis estimation processing executed by the determination device 100 will be described while referencing FIG. 2. In one example, the determination device 100 starts the body axis estimation processing when an instruction to start the body axis estimation processing is received, via the input device 41, from a user, namely, the subject.

First, the acceleration acquirer 11 of the determination device 100 acquires detection values (acceleration data) detected by the acceleration sensor of the sensor 30 (step S101). Typically, step S101 is continuously performed during a series of time periods during which the body motion of the subject is acquired. Examples of such time periods include from bedtime to wake-up time of the subject for whom the body axis is to be estimated. Then, the acceleration acquirer 11 stores, into the storage unit 20 in a time series (for example, every 25 milliseconds), the detection values detected during the series of time periods by the acceleration sensor of the sensor 30, together with information of the detected times.

In step S101, the acceleration acquirer 11 does not need to precisely know the series of time periods during which the body motion of the subject is to be acquired. Rather, it is sufficient that a timer settings (for example, from 23:00 to 7:00) are performed to acquire the detection values during the series of time periods. Additionally, the detection values during the series of time periods may be acquired based on instructions from the input device 41 (instructions to start and end the acquisition of the detection values). Step S101 is also called an “acceleration acquisition step.”

Next, the body motion determiner 12 performs the body motion determination processing (step S102). The body motion determination processing is processing for determining, based on the acceleration values, whether there is body motion of the subject. Details of this process will be described later. Step S102 is also called a “body motion determination step.”

Next, the evaluator 13 extracts, from the storage unit 20, the accelerations at the times at which the body motion determiner 12 determines that there is no body motion (step S103). Then, the evaluator 13 normalizes the extracted accelerations (step S104). The accelerations are normalized by dividing each element of the acceleration by the norm of that acceleration.

Next, the evaluator 13 performs acceleration distribution evaluation processing for the extracted and normalized accelerations (step S105). The acceleration distribution evaluation processing is processing for evaluating the manner of the distribution of the extracted accelerations. Details of this process will be described later. Step S105 is also called an “evaluation step.”

Next, the evaluator 13 determines, based on the determination results of whether there is a roll over (change in sleeping position) in the acceleration distribution evaluation processing, whether there is a roll over of the subject (step S106), When the estimator 14 determines that there is a roll over (step S106; Yes), the estimator 14 estimates the direction vector (the “eigenvector corresponding to the minimum eigenvalue” described later) in which the distribution is determined to be small in the acceleration distribution evaluation processing to be the body axis of the subject (step S107). When the estimator 14 determines that there is not a roll over (step S106; No), the estimator 14 estimates the direction vector (the “eigenvector corresponding to the maximum eigenvalue” described later) in which distribution is determined to be great in the acceleration distribution evaluation processing to be a vector orthogonal to the body axis of the subject (step S108). Step S107 and step S108 are also called “estimation steps.” After the estimation in step S107 and step S108, the body axis estimation processing is ended.

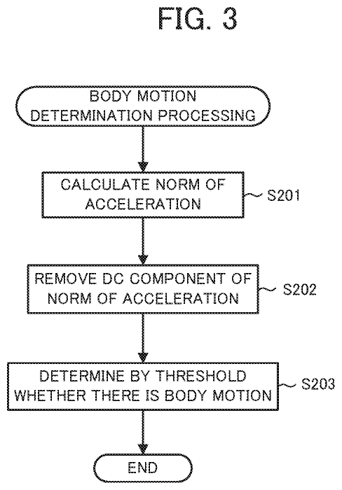

The body motion estimation processing is described above. The body motion determination processing executed in step S102 of the body motion estimation processing will be described while referencing FIG. 3.

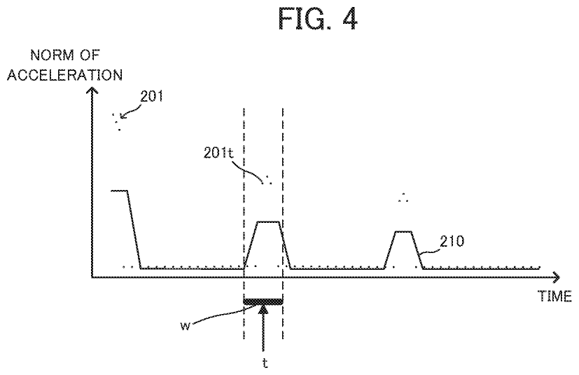

First, the body motion determiner 12 calculates the norm of acceleration for each of the accelerations acquired in step S101 of the body axis estimation processing and stored into the storage unit 20, and stores the calculated norms of acceleration into the storage unit 20 (step S201). The norms of acceleration calculated here form a data string such as illustrated by the dotted line of FIG. 4, for example. In the data string, the values fluctuate significantly only when there is a body motion of the subject.

Next, the body motion determiner 12 removes the DC components of the norms of acceleration calculated in step S201, and stores the results into the storage unit 20 (step S202). If the acceleration sensor is ideal (free of sensitivity variance and errors), the process for removing the DC component may simply include subtracting gravitational acceleration (1 G) from the norm of acceleration. However, in reality, due to the machining accuracy, sensitivity variance between axes, error, and the like the acceleration sensor, the body motion determiner 12 removes the DC component by subtracting the moving average from the norm of acceleration. In this case, the moving average is, as illustrated by line 210 in FIG. 4 for example, obtained by calculating the average of the norms of acceleration included in a predetermined time width window “w” centered on a time “t” (time of target norm of acceleration 201t) while moving the target norm of acceleration in the time direction. Then, the body motion determiner 12 removes the DC component of the norm of acceleration by subtracting the moving average at the same time from the norm of acceleration.

Note that various methods, other than subtracting the moving average at the same time from the norm of acceleration, may be used as the method for removing the DC component. Examples of such methods include obtaining the moving standard deviation in the same manner as the moving average, and regarding this moving standard deviation itself as the norm of acceleration from which the DC component has been removed. The body motion determiner 12 may obtain the moving standard deviation to remove the DC component from the norm of acceleration. An example of another method includes the body motion determiner 12 using a band pass filter (BPF) a high pass filter (HPF), or the like to remove the DC component of the norm of acceleration.

Next, the body motion determiner 12 compares the norm of acceleration from which the DC component has been removed with the predetermined body motion determination threshold obtained by experiment or the like to determine whether there is a body motion of the subject at the time corresponding to the norm of acceleration, and stores, into the storage unit 20, the results of determining whether there is a body motion together with time information (step S203). In step S203, it is preferable to determine, for all of the accelerations acquired in step S101, whether there is a body motion of the subject at the time corresponding to each acceleration. Then, the body motion determination processing is ended, and step S103 of the body axis estimation processing is executed.

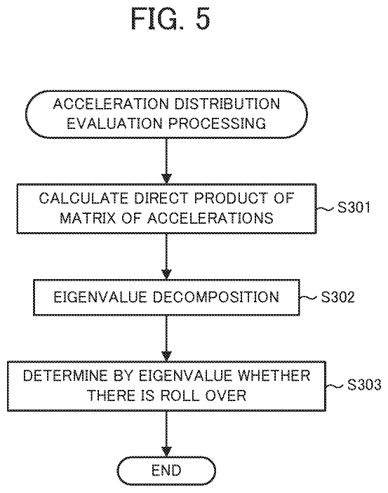

The body motion determination processing is described above. Next, the acceleration distribution evaluation processing executed in step S105 of the body motion estimation processing will be described while referencing FIG. 5.

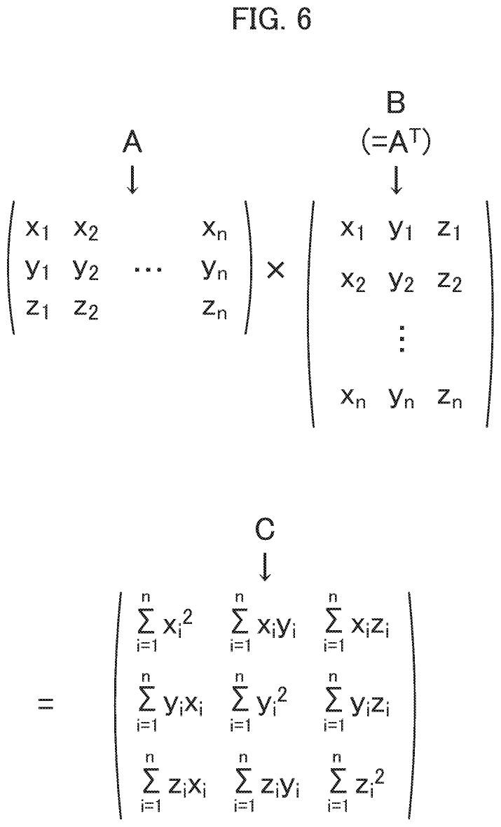

First, the evaluator 13 calculates the direct product of a matrix of vectors of the accelerations normalized in step S104 of the body axis estimation processing (step S301). In one example, it is assumed that n acceleration vectors (three-dimensional vectors with x, y, and z as elements) are extracted in step S103 of the body axis estimation processing. If the values obtained by normalizing the extracted acceleration vectors in step S104 are expressed as (x₁, y₁, z₁), (x₂, y₂, z₂), . . . , (xₙ, yₙ, zₙ), the direct product is calculated as illustrated in FIG. 6.

In FIG. 6, a matrix in which the normalized acceleration vectors (row vectors) are horizontally arranged is expressed as A, a matrix in which the normalized acceleration vectors (column vectors) are vertically arranged is expressed as B, and the direct product of A and B is expressed as C. It is clear from FIG. 6 that matrix B is a transposed matrix of matrix A, and that matrix C is a 3×3 symmetric matrix regardless of the value of “n” (the number of extracted acceleration vectors).

Next, the evaluator 13 performs an eigenvalue decomposition of the calculated direct product (matrix C in FIG. 6) to obtain an eigenvalue and an eigenvector corresponding to the eigenvalue (step S302). Then, the evaluator 13 determines, based on the obtained eigenvalue, whether there is a roll over of the subject (step S303), ends the acceleration distribution evaluation processing, and executes step S106 of the body axis estimation processing.

Next, a supplementary description will be given of the determination in step S303 of whether there is a roll over. Typical rolling over involves a rotational movement around the body axis. Accordingly, when there is a roll over, the distribution of acceleration in the body axis direction is extremely small, and accelerations in other directions are arranged on the circumference of a plane that has the body axis as the normal. That is, when there is a roll over, the minimum eigenvalue is sufficiently small, and the value of the second smallest eigenvalue is large compared to the minimum eigenvalue. Meanwhile, when there are no rolling over, little acceleration other than gravity acceleration is detected. That is, when there are no rolling over, the minimum eigenvalue is not very small, and the value of the second smallest eigenvalue is not much greater than the minimum eigenvalue. As such, the difference or ratio between the minimum eigenvalue and the second eigenvalue is smaller.

Accordingly, when, for example, the minimum eigenvalue is greater than a predetermined first threshold, the second smallest eigenvalue is less than a predetermined second threshold, and the ratio of the second smallest eigenvalue to the minimum eigenvalue is less than a predetermined third threshold, the evaluator 13 determines that there is not a roll over. In contrast, when, for example, the minimum eigenvalue is less than or equal to than the predetermined first threshold, the second smallest eigenvalue is greater than or equal to the predetermined second threshold, or the ratio of the second smallest eigenvalue to the minimum eigenvalue is greater than or equal to the predetermined third threshold, the evaluator 13 determines that there is a roll over. Appropriate values are set for these thresholds by having the subject actually attach the acceleration sensor and sleep, comparing the eigenvalues for cases when there is a roll over and when there is not a roll over, and the like.

Since the distribution of acceleration in the body axis direction is extremely small when there is a roll over, the direction vector in which the distribution is small, that is, the eigenvector corresponding to the minimum eigenvalue, is estimated to be the body axis. When there is not a roll over, the direction vector in which the distribution is great, that is, the eigenvector corresponding to the maximum eigenvalue, is estimated to be the vector orthogonal to the body axis.

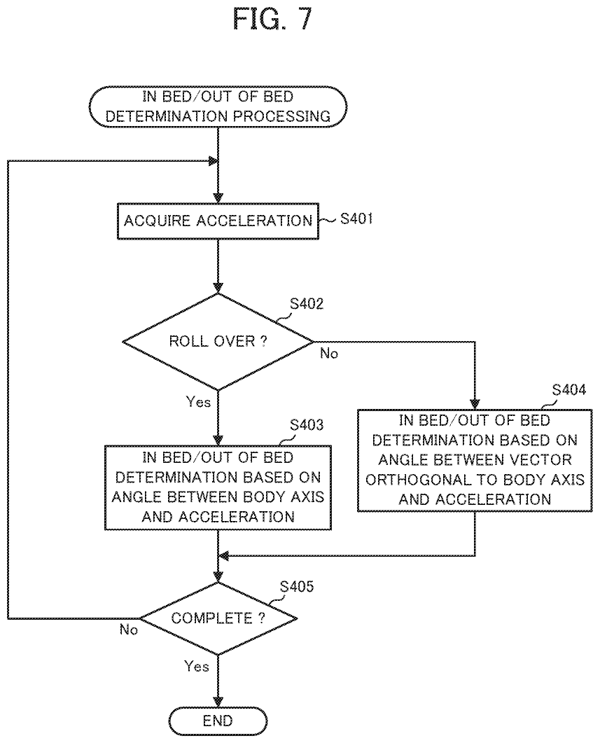

The estimation of the vector of the body axis of the subject or the vector orthogonal to the body axis is performed based on the body axis estimation processing, the body axis determination processing, and the acceleration distribution evaluation processing described above. Thus, the determination device 100 can perform determination processing for determining if the subject is in bed or is out of bed based on an angle difference between the estimated vector and the detection values from the acceleration sensor. In one example, the determination device 100 starts the determination processing when an instruction to start the determination processing is received from the user via the input device 41. However, in cases in which the body axis estimation processing has not already been executed, the body axis estimation processing is executed before starting the determination processing and, thereafter, the determination processing is started. The determination processing will be described while referencing FIG. 7.

First, the acceleration acquirer 11 of the determination device 100 acquires one acceleration in the time period for which in bed/out of bed determination is to be performed (step S401). Since, in step S101 of the body axis estimation processing, the acceleration data string is stored into the storage unit 20 together with the information of the time when each acceleration is acquired, the acceleration acquirer 11 acquires, from the storage unit 20, the accelerations, in the time period for which the in bed/out of bed determination is to be performed, one at a time along the time series.

Next, the determiner 15 determines whether the determination in step S106 of the body axis estimation processing (the determination in step S303 of the acceleration distribution evaluation processing) is that there is a roll over (step S402). When the determiner 15 determines that there is a roll over (step S402; Yes), the determiner 15 determines that the subject is in bed if a state in which the angle, formed between the body axis estimated in the body axis estimation processing and the acceleration acquired in step S401, is less than a predetermined determination threshold (for example, 45 degrees) continues for a predetermined amount of time (for example, one minute), and determines that the subject is out of bed if the angle is greater than or equal to the determination threshold (step S403). Meanwhile, when the determiner 15 determines that there is not a roll over (step S402; No), the determiner 15 determines that the subject is out of bed if an angle, formed between the vector orthogonal to the body axis estimated in the body axis estimation processing and the acceleration acquired in step S401, is less than a predetermined determination threshold (for example, 45 degrees), and determines that the subject is in bed if a state in which the angle is greater than or equal to the determination threshold continues for a predetermined amount of time (for example, one minute) (step S404).

Step S403 and step S404 are also called “determination steps.” When performing the in bed/out of bed determination in step S403 and step S404, it is possible to determine using two thresholds to provide hysteresis instead of simply using only one threshold. For example, in a case in which there is a roll over, the determiner 15 determines that the subject is in bed when it is determined immediately before that the subject is in bed and if a state, in which the angle described above is less than a first determination threshold (for example, 60 degrees), continues for a predetermined amount of time (for example, one minute); determines that the subject is in bed when it is determined immediately before that the subject is out of bed and if a state, in which the angle described above is less than a second determination threshold (for example, 30 degrees), continues for a predetermined amount of time (for example, one minute); and determines that the subject is out of bed if the angle described above is greater than or equal to the second threshold.

Additionally, when performing the in bed/out of bed determination in steps S403 and step S404, chattering removal processing, determination processing including a state transition for each predetermined amount of time, and the like may be performed. The chattering removal processing is a process in which, when situations occur in which the in bed/out of bed determination switches in a short amount of time, the intermediate switching is disregarded and only the final determination results are used. The determination processing involving a state transition for each duration is a process in which the subject is not considered to be out of bed unless continuously determined to be out of bed for a predetermined amount of time (for example, 10 minutes). With such a configuration, it is possible to distinguish between whether the subject has gotten out of bed or is merely temporarily out of bed to go to the bathroom, for example.

After the in bed/out of bed determination described above, the determiner 15 determines whether a completion condition is satisfied (step S405). Any condition can be set as the completion condition. For example, the completion condition may be that all of the accelerations in the time period for which in bed/out of bed determination is to be performed are performed, or that an end instruction is input from the input device 41.

If it is determined that the completion condition is not satisfied (step S405; No), step S401 is executed. If it is determined that the completion condition is satisfied (step S405; Yes), the determination processing is ended.

According to the body axis estimation processing and the determination processing described above, the in bed/out of bed determination is performed after appropriately estimating the body axis (or the vector orthogonal to the body axis). As such, it is possible to appropriately determine if the subject is in bed or out of bed regardless of the attached location and attached direction of the acceleration sensor, the body motions of the subject, and the like.

### MODIFIED EXAMPLE 1

In the embodiment described above, whether there is a roll over of the subject is determined, and the processing content is changed depending on whether there is a roll over. However, since it is extremely rare for there to be no rolling over, in many cases it is possible to omit the determination of whether there is a roll over. As such, in Modified Example 1, a case is described in which the roll over determination processing is omitted.

In the body axis estimation processing according to Modified Example 1, step S106 and step S108 are deleted, and the processing of step S107 is performed after the processing of step S105. Additionally, in the acceleration distribution evaluation processing according to Modified Example 1, step S303 is deleted and, after the processing of step S302, the processing of step S107 of the body axis estimation processing is performed. Moreover, in the determination processing according to Modified Example 1, the processing of step S402 and step S404 is omitted, and the processing of step S403 is performed after the processing of step S401.

Even when there is not a clear roll over, the acceleration distribution in the body axis direction is always extremely small. As such, in many cases, there is no problem estimating the eigenvector corresponding to the minimum eigenvalue to be the body axis and, in most cases, the body axis is appropriately estimated.

Thus, with the determination device 100 according to Modified Example 1, the processing load is reduced an amount corresponding to the roll over determination processing that is deleted. Moreover, with the determination device 100 according to Modified Example 1, the in bed/out of bed determination is performed after appropriately estimating the body axis. As such, it is possible to appropriately determine if the subject is in bed or out of bed regardless of the attached location and attached direction of the acceleration sensor, the body motions of the subject, and the like. Additionally, even when the roll over determination processing is omitted, the subject rolls over in most cases and, even when there is not a clear roll over, the acceleration distribution in the body axis direction is extremely small. As such, even though the processing load is decreased, the effect on the accuracy of the in bed/out of bed determination is negligible.

### MODIFIED EXAMPLE 2

In the embodiment described above, an eigenvalue decomposition of the direct product was performed when evaluating the acceleration distribution. However, a configuration is possible in which a singular value decomposition of the direct product is performed instead of the eigenvalue decomposition. Next, Modified Example 2, in which singular value decomposition is carried out, will be described.

The evaluator 13 according to Modified Example 2 extracts, from among the acceleration data string acquired by the acceleration acquirer 11, the accelerations for which the body motion determiner 12 determines that there is no body motion, performs a singular value decomposition of a matrix of the extracted accelerations, and calculates singular values and a singular vector corresponding to each of the singular values. The singular values and singular vectors are equivalent to the eigenvalues and the eigenvectors in Embodiment 1 described above, and are values in which features (the acceleration distribution) of the motion of the subject are reflected.

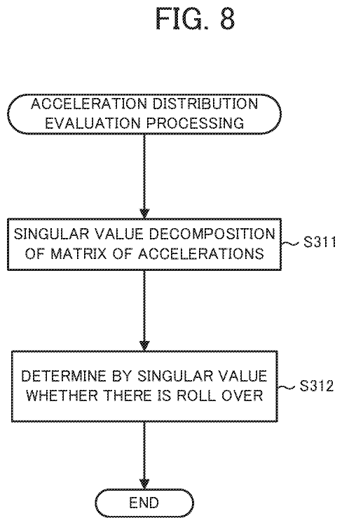

The body axis estimation processing according to Modified Example 2 is fundamentally the same as that of Embodiment 1 but, the acceleration distribution evaluation processing executed in step S105 differs. As such, the acceleration distribution evaluation processing according to Modified Example 2 will be described while referencing FIG. 8.

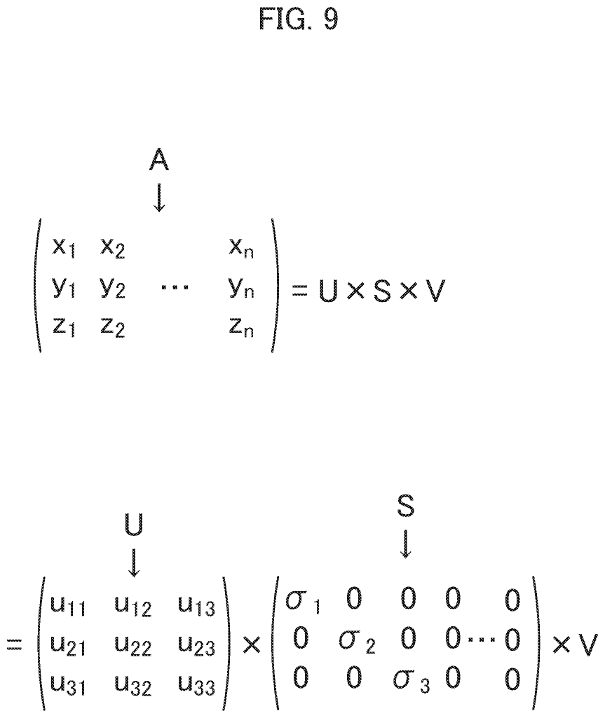

First, the evaluator 13 performs a singular value decomposition of a matrix of vectors of the accelerations normalized in step S104 of the body axis estimation processing. In one example, it is assumed that n acceleration vectors (three-dimensional vectors with x, y, and z as elements) are extracted in step S103 of the body axis estimation processing. The values obtained by normalizing the extracted acceleration vectors in step S104 are expressed as (x₁, y₁, z₁), (x₂, y₂, z₂), . . . , (xₙ, yₙ, zₙ). The phrase “singular value decomposition” means, as illustrated in FIG. 9, decomposing the matrix A, in which the normalized acceleration vectors (line vectors) are horizontally arranged, into the form of matrix U×matrix S×matrix V.

In FIG. 9, the matrix U is a 3×3 orthogonal matrix, and matrix V is an “n”×“n” orthogonal matrix. Matrix S is a 3×n matrix in which the off-diagonal components are 0 and the diagonal components are non-negative and arranged in order of magnitude. It is mathematically proven that the singular value decomposition described above can be performed on any matrix. The term “singular value” refers to the diagonal components (σᵢ) of the matrix S. Additionally, each row (row vector) of the matrix U is referred to as a singular vector, and corresponds to the singular value with the same row number. For example, the singular vector that corresponds to the singular value (σ₁) is (u₁₁, u₂₁, u₃₁)T (the right-side “T” represents transposition).

Then, the evaluator 13 determines, based on the singular value obtained through the singular value decomposition, whether there is a roll over of the subject (step S312), ends the acceleration distribution evaluation processing, and executes step S106 of the body axis estimation processing.

The determination of whether there is a roll over in step S312 is similar to the determination of whether there is a roll over in step S303 of the acceleration distribution evaluation processing of Embodiment 1. However, in the supplementary description of the determination of whether there is a roll over in step S303, mentions of “eigenvalue” must be replaced with “singular value”, and mentions of “eigenvector” must be replaced with “singular vector.” The singular value decomposition is substantially equivalent to the eigenvalue decomposition of the direct product and, as such, the acceleration distribution evaluation processing according to Modified Example 2 is equivalent to the acceleration distribution evaluation processing according to Embodiment 1.

Accordingly, with the determination device 100 according to Modified Example 2, similar to the determination device 100 according to Embodiment 1, the in bed/out of bed determination is performed after appropriately estimating the body axis (or the vector orthogonal to the body axis). As such, it is possible to appropriately determine if the subject is in bed or out of bed regardless of the attached location and attached direction of the acceleration sensor, the body motions of the subject, and the like.

### OTHER MODIFIED EXAMPLES

In the embodiment described above, an acceleration sensor for detecting acceleration is provided as the sensor 30. However, in cases in which acceleration information can be acquired from an external device or the like via the communicator 43, the determination device 100 need not include the sensor 30. Additionally, in the embodiment described above, the determination device 100 includes the input device 41, the output device 42, and the communicator 43. However, these are not essential constituents and configurations are possible in which the determination device 100 does not include the input device 41, the output device 42, and/or the communicator 43.

In the embodiment and the modified examples described above, eigenvalue decomposition of the direct value or singular value decomposition is performed to evaluate the acceleration distribution. However, the method for evaluating the acceleration distribution is not limited thereto. Any method can be used to evaluate the acceleration vector distribution, provided that it is possible to obtain the vectors that are distributed at frequently and the vectors that are distributed only infrequently.

Moreover, in the embodiment and the modified examples described above, the in bed/out of bed determination of the subject was performed. However, a configuration is possible in which in bed and/or out of bed determination is performed.

Note that, the various functions of the determination device 100 can be implemented by a computer such as a typical personal computer (PC). Specifically, in the embodiment described above, an example is described in which the programs, such as the body axis estimation processing and the determination processing, performed by the determination device 100 are stored in advance in the ROM of the storage unit 20. However, a computer may be configured that is capable of realizing these various features by storing and distributing the programs on a non-transitory computer-readable recording medium such as a compact disc read-only memory (CD-ROM), a digital versatile disc (DVD), a magneto-optical disc (MO), a memory card, and universal serial bus (USB) memory, and reading out and installing these programs on the computer.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

What is claimed is:

1. A determination device comprising at least one processor,
the processor being configured to:
acquire, in a time series, information about a body motion of a subject from a sensor attached by the subject;
determine, based on the acquired information about the body motion, whether there is a body motion of the subject at each time, evaluate a distribution of the information about the body motion of the subject at a time at which it is determined that there is not a body motion, and estimate a body axis of the subject based on the evaluated distribution of the information about the body motion; and
determine at least one of in bed and out of bed of the subject based on the estimated body axis of the subject.

2. The determination device according to claim 1, wherein:
the sensor includes an acceleration sensor, and
the information about the body motion of the subject is an acceleration acquired from the acceleration sensor.

3. The determination device according to claim 2, wherein:
the processor is configured to:
calculate a norm of the acquired acceleration, remove a DC component from the calculated norm of the acceleration, and determine whether there is a body motion of the subject based on the norm of the acceleration from which the DC component has been removed.

4. The determination device according to claim 2, wherein:
the processor is configured to:
estimate, based on the evaluated distribution of the acceleration, a direction vector in which the distribution of the acceleration is small to be a direction of the body axis of the subject.

5. The determination device according to claim 4, wherein:
the processor is configured to
calculate a direct product of a matrix generated from accelerations at times at which it is determined that there is not a body motion of the subject, perform an eigenvalue decomposition of the calculated direct product, and set an eigenvector corresponding to a minimum eigenvalue obtained by the eigenvalue decomposition as the direction vector in which the distribution of the acceleration is small.

6. The determination device according to claim 4, wherein:
the processor is configured to:
evaluate the distribution of the acceleration by performing singular value decomposition of a matrix generated from accelerations at times at which it is determined that there is not a body motion of the subject, and
estimate a singular vector corresponding to a minimum singular value obtained by the singular value decomposition to be the direction of the body axis of the subject.

7. The determination device according to claim 2, wherein:
the processor is further configured to:
determine that the subject is in bed when a state, in which an angle formed between the estimated body axis of the subject and the acquired acceleration is less than a predetermined determination threshold, continues for a predetermined amount of time.

8. The determination device according to claim 2, wherein:
the processor is configured to determine, based on the evaluated distribution of the acceleration, whether there is a roll over of the subject, and
when it is determined that there is a roll over, estimate a direction vector in which the distribution of the acceleration is small to be the body axis of the subject, and
when it is determined that there is not a roll over, estimate a direction vector in which the distribution of the acceleration is great to be a direction orthogonal to the body axis of the subject.

9. The determination device according to claim 8, wherein:
the processor is further configured to calculate a direct product of a matrix generated from accelerations at times at which it is determined that there is not a body motion of the subject, and evaluate the distribution of the acceleration by eigenvalue decomposing the calculated direct product, and
when it is determined that there is a roll over, estimate an eigenvector corresponding to a minimum eigenvalue obtained by the eigenvalue decomposition to be a direction vector of the body axis of the subject, and
when it is determined that there is not a roll over, estimate an eigenvector corresponding to a maximum eigenvalue obtained by the eigenvalue decomposition to be a direction vector orthogonal to the body axis of the subject.

10. The determination device according to claim 8, wherein:
the processor is further configured to evaluate the distribution of the acceleration by performing singular value decomposition of a matrix generated from accelerations at times at which it is determined that there is not a body motion of the subject, and
when it is determined that there is a roll over, estimate a singular vector corresponding to a minimum singular value obtained by the singular value decomposition to be a direction vector of the body axis of the subject, and
when it is determined that there is not a roll over, estimate a singular vector corresponding to a maximum singular value obtained by the singular value decomposition to be a direction vector orthogonal to the body axis of the subject.

11. The determination device according to claim 8, wherein:
the processor is configured to:
when it is determined that there is a roll over,
determine that the subject is in bed when a state, in which an angle formed between the estimated body axis of the subject and the acquired acceleration is less than a predetermined determination threshold, continues for a predetermined amount of time, and
when it is determined that there is not a roll over,
determine that the subject is in bed when a state, in which an angle formed between a direction vector orthogonal to the estimated body axis of the subject and the acquired acceleration is greater than or equal to a predetermined determination threshold, continues for a predetermined amount of time.

12. A determination method comprising:
acquiring, in a time series, information about a body motion of a subject;
determining, based on the acquired information about the body motion, whether there is a body motion of the subject at each time, evaluating a distribution of the information about the body motion of the subject at a time at which it is determined that there is not a body motion, and estimating a body axis of the subject based on the evaluated distribution of the information about the body motion; and
determining at least one of in bed and out of bed of the subject based on the estimated body axis of the subject.

13. A non-transitory computer-readable recording medium on which a program is recorded, the program causing a computer of a determination device to:
acquire, in a time series, information about a body motion of a subject;
determine, based on the acquired information about the body motion, whether there is a body motion of the subject at each time, evaluate a distribution of the information about the body motion of the subject at a time at which it is determined that there is not a body motion, and estimate a body axis of the subject based on the evaluated distribution of the information about the body motion; and
determine at least one of in bed and out of bed of the subject based on the estimated body axis of the subject.
